(19) United States
(12) Patent Application Publication
(10) Pub. No.: US 2020/0121586 A1
(43) Pub. Date: Apr. 23, 2020

(54) COMPOSITION COMPRISING CHARCOAL AND AN EXTRACT OR SUBSTANCE OF ASTROCARYUM MURUMURU

(71) Applicant: Procarbon LLC, Jupiter, FL (US)

(72) Inventor: Martin Giniger, Pompano Beach, FL (US)

(21) Appl. No.: 16/656,674

(22) Filed: Oct. 18, 2019

Related U.S. Application Data
(60) Provisional application No. 62/747,923, filed on Oct. 19, 2018.

(51) Int. Cl. A61K 8/9794; A61K 8/96; A61K 8/22; A61Q 11/00

(52) U.S. Cl. CPC A61K 8/9794; A61Q 11/00; A61K 8/22; A61K 8/96

(57) ABSTRACT

A composition and methods of using the composition to promote oral cavity health. The oral composition comprises charcoal and plant substance or extract for use in oral care, particularly in teeth cleaning and maintenance. The oral composition is made up of multiple ingredients, including one or more of charcoal, a botanical activator, a whitening agent, solvent, an abrasive, a thickening agent, a secondary thickening agent, a foaming agent, a humectant, a colorant, and a flavorant. The multiple ingredients may be mixed together in the form of a toothpaste for delivery to an individual's teeth. The oral composition may be formulated as two components which are separated from each other until use, i.e. brushing of teeth.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 62/747,923, entitled “COMPOSITION COMPRISING CHARCOAL AND AN EXTRACT OR SUBSTANCE OF ASTROCARYUM MURUMURU”, filed Oct. 19, 2018. The contents of the above referenced application are incorporated herein by reference.

### FIELD OF THE INVENTION

The present invention relates to a composition comprising charcoal and a plant substance or extract; to an oral composition comprising charcoal and plant substance or extract; and more particularly, to a composition comprising an activated charcoal and Astrocaryum Murumuru substances or extracts for use in the oral cavity, such as for cleaning teeth, and to dental cleaning compositions comprising an activated charcoal and substances or extracts of Astrocaryum Murumuru.

### BACKGROUND OF THE INVENTION

Proper dental hygiene is not only vital for healthy teeth, but also for the maintenance of one's overall health. Proper dental cleaning may help lower the risk of heart disease and stroke, and may prevent oral cancer and gum disease. It is recommended that individuals brush their teeth at least two times a day. Additional brushing may be required, particularly as a result of the many ingredients that are used in food products that promote tooth decay, such as refined sugars found in junk foods and soda drinks. Use of a good oral composition, such as toothpaste, is an important part of an individual's dental health and maintenance routine.

### SUMMARY OF THE INVENTION

The present invention relates to a composition, and methods of using the composition, to promote oral cavity health. The present invention relates to an oral composition comprising charcoal and plant substance or extract for use in oral care, particularly in teeth cleaning and maintenance. The oral composition is made up of multiple ingredients, including one or more of charcoal, a botanical activator, a whitening agent, solvent, an abrasive, a thickening agent, a secondary thickening agent, a foaming agent, a humectant, a colorant, and a flavorant. The multiple ingredients may be mixed together in the form of a toothpaste for delivery to an individual's teeth. The oral composition may be formulated as two components which are separated from each other until use, i.e. brushing of teeth.

Accordingly, it is an objective of the invention to provide a composition comprising charcoal and plant substance or extract.

It is a further objective of the invention to provide an oral composition comprising charcoal and plant substance or extract in the form of a toothpaste for delivery to the oral cavity.

It is yet another objective of the invention to provide a two-component oral composition comprising charcoal, chemical whitening agents, and a plant substance or extract activator.

It is a still further objective of the invention to provide an oral composition comprising charcoal, chemical whitening agents, and a plant substance or extract activator of which two components are separated until use.

It is a still further objective of the invention to provide a two-component oral composition in which the first component or the second component include charcoal, chemical whitening agents, a plant substance or extract activator, solvent, an abrasive, a thickening agent, a secondary thickening agent, a foaming agent, a humectant, a colorant, or flavorant.

It is a still further objective of the invention to provide a two-component oral composition in which the first component or the second component include charcoal, chemical whitening agents, a plant substance or extract activator, solvent, an abrasive, a thickening agent, a secondary thickening agent, a foaming agent, a humectant, a colorant, or flavorant are stored separately, and when mixed together are capable of being delivered to the oral cavity and providing various teeth cleaning and maintenance functionalities.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a schematic representation of an illustrative embodiment of a dual chamber toothpaste dispenser.

### DETAILED DESCRIPTION OF THE INVENTION

In certain embodiments of the present invention, a plurality of ingredients is combined to form a unique composition. Preferably, the present invention is a combinable, two component oral composition (a first oral composition component preferably containing charcoal and an chemical whitening agent, and a second oral composition component containing a plant based actuator), also referred to herein as a dual component oral composition, in the form of a toothpaste, for use in cleaning teeth. The dual component oral composition is most preferably a whitening toothpaste or teeth whitening gel. The two components are separated from each other until use, i.e. brushing of teeth. Accordingly, the oral composition is made up of various ingredients which are capable of being mixed together in the form of a toothpaste for delivery to the oral cavity, and for providing various teeth cleaning and maintenance functionalities. While the oral composition is preferably formulated as a whitening toothpaste, other means of delivery, such as gels or liquids, can be formulated.

The oral composition preferably comprises charcoal, a botanical activator, a whitening agent, and one or more other ingredients, including a solvent, an abrasive, a thickening agent, a secondary thickening agent, a foaming agent, a humectant, a colorant, and a flavorant.

In some embodiments, the first oral composition component or the second oral composition component includes charcoal, preferably activated charcoal. Activated charcoal is a form of carbon processed to have small, low-volume pores that increase the surface area available for adsorption or chemical reactions. Activated charcoal is typically made by heating common charcoal in the presence of a gas, causing the charcoal to develop lots of internal spaces or pores. These pores help activated charcoal trap chemicals.

In some embodiments, the oral composition includes a botanical activator. As used herein, a “botanical activator” is a plant based substance which can react with one or more components of the oral composition to produce oxygen free radicals. The preferred botanical activator is Astrocaryum Murumuru, including Murumuru butter. Astrocaryum Murumuru is a palm found in Brazil and other regions of the Amazon. The Murumuru palm trees have a thick trunk and a shuttlecock-shaped bushy crown, and bear an edible fruit. The fruit contains a white butter that is odorless and tasteless. Murumuru butter contains lauric, myristic, and oleic acids.

Murumuru reacts with calcium peroxide, and water from saliva, to cause breakdown into calcium and oxygen (O—) free radicals. The free radicals are free to whiten teeth. Calcium peroxide is a solid chemical used as a source of oxygen or hydrogen peroxide. When placed in water, calcium peroxide begins to decompose and release oxygen. The calcium peroxide is kept separated from the Murumuru, until actual use, i.e. the consumer is ready to brush their teeth. When both components are dispensed on the toothbrush together, the Murumuru and Calcium Peroxide combine for the first time, and then start to foam and whiten when the entire mixture comes into contact with water from saliva.

In some embodiments, the oral composition includes whiteners. Suitable non-limiting representatives of whiteners may include peroxides, such as calcium peroxide. The whitening or bleaching agent may be selected from hydrogen peroxide (H₂O₂), or any compound that yields hydrogen peroxide when placed in aqueous medium, such as carbamide peroxide (also known as urea peroxide, urea hydrogen peroxide, hydrogen peroxide carbamide, and perhydrol urea).

In some embodiments, the first oral composition component or the second oral composition component includes a solvent(s). Suitable non-limiting representatives of solvents may include alkaline water. As indicated, the vehicle is preferably aqueous, but it is within the broader scope of the invention to employ non-aqueous vehicles. Generally, the liquid will contain a humectant or other viscous water-miscible material such as glycerol, sorbitol, polyethylene glycol, mannitol or mixtures thereof. When water is present, it preferably constitutes about 5% to 35% (e.g. about 10% to 30%) of the total vehicle. As used herein, the term “about” can be defined as any value that is effective for its function or a value which is 10%-20% above or below the stated concentration. Superior results (such as better taste) are obtained when the proportion of water is relatively low, e.g. about 10% to 20% of the total toothpaste, such as when the water to glycerol ratio is in the range of about 0.4:1 to 0.7:1.

In some embodiments, the first oral composition component or the second oral composition component includes abrasive(s). Suitable non-limiting representatives of abrasives may include silica abrasives. The oral composition may contain from about 6% to 45%, preferably from about 10% to 30%, by weight of a silica abrasive polishing material. Silica dental abrasives of various types can provide the unique benefits of exceptional dental cleaning and polishing performance without unduly abrading tooth enamel or dentin. Silica abrasive materials are also exceptionally compatible with sources of soluble fluoride. The silica abrasive polishing material used herein generally has an average particle size ranging between about 0.1 to 30 microns, preferably 5 and 15 microns. The abrasive can be precipitated silica or silica gels, such as the silica xerogels described in U.S. Pat. No. 3,538,230, and incorporated herein by reference. Preferred are the silica xerogels marketed under the tradename SIDENT 9 (W. R. Grace & Company, Davison Chemical Division). Precipitated silica materials, such as those marketed by the J. M. Huber Corporation under the tradename, ZEODENT, particularly the silica carrying the designation ZEODENT 119, may be used as well. Other types of silica dental abrasives useful in the oral composition of the present invention are described U.S. Pat. No. 3,862,307 and incorporated herein by reference.

In some embodiments, the first oral composition component or the second oral composition component includes thickening agent(s). Suitable non-limiting representatives of thickening agents may include gelling agents for toothpaste vehicles, as are well known in the art. These are often high polymers (e.g. gums or other thickening agents) which are soluble or swellable in water or aqueous medium. The thickening agent may be Sodium carboxy methylcellulose, gum tragacanth, gum arabic, gum karaya, sodium alginate, hydroxyethyl cellulose, methyl cellulose, ethyl cellulose, carrageen and other polysaccharides, polyvinyl pyrrolidones. Other thickening agents may include VEEGUM, a complex magnesium aluminum silicate. The amount of thickening agent used in the oral composition is preferably sufficient to impart to the mixture a pasty consistency, body and the non-tacky nature which is characteristic of conventional dental creams or toothpastes. Such dental creams are extrudable from ordinary collapsible toothpaste tubes to form a ribbon of substantial thickness (e.g. about ⅜ inch) which, if left undisturbed, substantially retains its original thickness over a period of, for example, one minute or more (and does not penetrate substantially into the bristles of a toothbrush when resting on the end of such bristles for a similar period); but which preferably offers no substantial resistance to brushing or to deformation when, for instance, one touches it lightly with a finger; and which has little tack, in that it does not tend to form a string when the finger is pulled away from the ribbon. The proportion of thickening agent is often within the range of about 0.5% to 2%, such as about 0.8% to 1.5%, of the toothpaste.

Silica thickener: Gel toothpastes generally contain up to 8.5 wt % thickening silica, whereas opaque toothpastes typically contain 3 to 4 wt % thickening silica. Preferred toothpaste compositions may include up to 2 wt % thickening silica. Further embodiments have 1.5 wt %, or even less than 1 wt % thickening Silica, such as less than 0.5 wt % thickening silica. Highly preferred compositions are free of thickening silica. When present, preferred thickening silicas include silica gels such as SYLODENT or SIDENT series (W. R. Grace & Company), or precipitated silica such as ZEOTHIX 265 (J. M. Huber Corporation). AEROSIL-T series (Degussa) or the CAB-O-SIL series (Cabot Corporation) can be used as well. Useful silica thickeners may also include ZEODENT 165, ZEODENT 163 and/or 167 and ZEOFREE® 153, 177, and/or 265 silicas, (J. M. Huber Corporation). Other thickening silicas include MFIL, MFIL-P (Madhu Silica, India), SIDENT 22 S and AEROSIL 200 (Evonik Industries), SYLODENT and SIDENT thickening silicas from W. R. Grace & Company and TAXOSIL T43 and 331 (Rhodia).

In some embodiments, the first oral composition component or the second oral composition component includes secondary thickening agent(s). The secondary thickening agent may include, but is not limited to, Irish moss, iota-carrageenan, kappa-carrageenan, gum tragacanth, starch, polyvinylpyrrolidone, hydroxyethyl propyl cellulose, hydroxybutyl methyl cellulose, hydroxypropyl methyl cellulose, hydroxyethyl cellulose and sodium carboxymethyl cellulose, or any combination thereof.

In some embodiments, the first oral composition component or the second oral composition component includes humectant(s). The humectant serves to keep the oral composition components from hardening upon exposure to air. Certain humectants can also impart desirable sweetness or flavor to the oral composition. The humectant, on a pure humectant basis, generally comprises from about 30% to 70%, preferably from about 45% to 65%, by weight of the oral compositions herein. Suitable non-limiting representatives of humectants include edible polyhydric alcohols such as glycerin, sorbitol, xylitol and propylene glycol. Sorbitol is frequently employed as a 70% aqueous solution known as SOROBO. Mixtures of glycerin and sorbitol may be preferred as the humectant component of the oral composition.

In some embodiments, the first oral composition component or the second oral composition component includes a foaming agent(s) (surface active agent(s)). An organic surface active agent is preferably used in the oral composition of the present invention to aid in the prophylactic action in the thorough dispersion of the composition throughout the oral cavity, and to improve cosmetic acceptability and detersive and foaming properties. Illustrative examples include water-soluble salts of the higher alkyl sulfates, such as sodium lauryl sulfate or other suitable alkyl sulfate having 8 to 18 carbon atoms in the alkyl group; water-soluble salts of sulfonated monoglycerides of higher fatty acids, such as sodium coconut monoglyceride sulfonate or other suitable sulfonated monoglyceride of a fatty acid of 10 to 18 carbon atoms; salts of amides of higher fatty acid (e.g. 12 to 16 carbon atom acids) with lower aliphatic amino acids (e.g. taurine or sarcosine) or other amino acids of 2 to 6 carbon atoms, such as sodium-N-methyl-N-palmitoyl taurine, sodium N-lauroyl, N-myristoyl or N-palmitoyl sarcosinates; water-soluble salt of the esters of such fatty acids with isethionic acid or with glycerol monosulfate, such as the sodium salt of the monosulfated monoglyceride of hydrogenated coconut oil fatty acids; water-soluble salts of olefin sulfonates, e.g. alkene sulfonates or hydroxyalkane sulfonates or mixtures thereof having 12 to 18 carbon atoms in the carbon chain of the molecule; water-soluble soaps of higher fatty acids, such as those of 12-18 carbon atoms, e.g. coconut fatty acids. The cation of the salt may be, for instance, sodium (which is preferred), potassium or mono- di- or triethanolamine. Mixtures of surface active agents may be used. A particularly suitable mixture, which provides a high foaming powder with little or no irritating effect, comprises a higher alkyl sulfate and a higher fatty acid sarcosinate, e.g. in a ratio of about 1:2 to 2:1, such as about 1:1; instead of all or part of the sarcosinate, a higher fatty acid monoglyceride sulfonate may be present.

Other suitable surface-active materials include nonionic agents such as condensates of sorbitan monostearate with approximately 60 of ethylene oxide, condensates of ethylene oxide with propylene oxide condensates of propylene glycol (available under the trademark PLURONICS) and amphoteric agents such as quaternized imidazole derivatives, which are available under the trademark MIRANOL, such as MIRANOL C2 M. Cationic surface-active germicides and antibacterial compounds may also be used. Such compounds include di-isobutylphenoxyethyoxyethyl dimethyl benzyl ammonium chloride, benzyl dimethyl stearyl ammonium chloride, tertiary amines, having one fatty alkyl group (of from 12 to 18 carbon atoms) and two (poly) oxyethylene groups attached to the nitrogen (typically containing a total of from about 2 to 50 ethenoxy groups per molecule) and salts thereof with acids, and compounds of the structure:

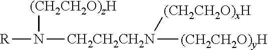

where R is a fatty alkyl group consisting from about 12 to 18 carbon atoms, and x, y and z total a number of 3 or higher, as well as salts thereof with mineral or organic acids. It is preferred to use from about 0.05 to 5 percent by weight of the foregoing surface-active materials in the instant oral composition preparations. The proportion of surface-active agent is preferably within the range of about 0.1% to 10% of the toothpaste, more preferably in the range of about 1% to 3%, such as about 1½% to 2%.

In some embodiments, the first oral composition component or the second oral composition component includes flavoring agent(s). Suitable non-limiting representatives of flavoring agents may include any suitable flavoring or sweetening materials, such as flavoring oils, for example, oils of spearmint, peppermint, wintergreen, sassafras, clove, sage, eucalyptus, marjoram, cinnamon, lemon and orange, as well as methyl salicylate. Suitable sweetening agents include xylitol, lactose, maltose, sorbitol, sodium cyclamate, perillartine, saccharine and ammoniated glycyrrhizin (e.g. its monoammonium salt). Suitably, the flavoring and sweetening agent together may comprise from about 0.01 to 50 percent, or more, of the compositions of the oral composition. Preferably, the amount of flavoring oil is above 0.4%, e.g. 0.81% to 5%.

In some embodiments, the first oral composition component or the second oral composition component includes coloring agent(s). Suitable non-limiting representatives of coloring agents may include titanium dioxide (TiO₂). The weight of titanium dioxide particles in the toothpaste is generally above about 0.9% of the weight of the toothpaste. The amount of TiO₂ may be included in amounts up to about 6.0%, preferably about 0.2% to 0.6% of the weight of the toothpaste. The particle size of the TiO₂ is preferably about 0.1 to 1 micron.

The following examples are illustrative embodiments of the dual component oral composition in accordance with the present invention. While examples provided herein describe the dual component in the preferred embodied form of a toothpaste, the dual component may be formulated in other forms, such as, but not limited to, gel or liquid formulations.

Dual Component Oral Composition: Example 1

| First Component | Second Component |
|---|---|
| Solvent | Solvent |
| Charcoal | Botanical Activator |
| Chemical Whitener | |

Dual Component Composition: Example 2

| First Component | Second Component |
|---|---|
| Solvent | Solvent |
| Charcoal | Astrocaryum Murumuru |
| Chemical Whitener | |

Dual Component Composition: Example 3

| | |
|---|---|
| First Component | Second Component |
| Solvent | Solvent |
| Charcoal | Astrocaryum Murumuru |
| Chemical Whitener | |

Dual Component Oral Composition: Example 4

| | |
|---|---|
| First Component | Second Component |
| Solvent | Solvent |
| Charcoal | Astrocaryum Murumuru |
| Calcium Peroxide | |

Dual Component Oral Composition: Example 5

| First Component | Second Component |
|---|---|
| Solvent | Solvent |
| Charcoal | Astrocaryum Murumuru |
| Calcium Peroxide | Other ingredients, as required |
| Other ingredients, | |
| as required | |

Dual Component Oral Composition: Example 6

| First Component | Second Component |
|---|---|
| Solvent | Solvent |
| Charcoal | Botanical Activator |
| Abrasive | Abrasive |
| Peroxide | Thickening Agent |
| Thickening Agent | Humectant |
| Humectant | |

Dual Component Oral Composition: Example 7

| First Component | Second Component |
|---|---|
| Solvent | Solvent |
| Charcoal | Botanical Activator |
| Abrasive | Abrasive |
| Peroxide | Thickening Agent |
| Thickening Agent | Humectant |
| Humectant | Foaming Agent |
| Foaming Agent | |

Dual Component Oral Composition: Example 8

| | |
|---|---|
| First Component | Second Component |
| Solvent | Solvent |
| Charcoal | Botanical Activator |
| Abrasive | Abrasive |
| Peroxide | Thickening Agent |
| Thickening Agent | Humectant |
| Humectant | Foaming Agent |
| Foaming Agent | Colorant |
| Colorant | |

Dual Component Oral Composition: Example 9

| First Component | Second Component |
|---|---|
| Solvent | Solvent |
| Charcoal | Botanical Activator |
| Abrasive | Abrasive |
| Peroxide | Thickening Agent |
| Thickening Agent | Humectant |
| Humectant | Foaming Agent |
| Foaming Agent | Secondary thickener |
| Secondary thickener | Colorant |
| Colorant | Flavorant |
| Flavorant | |

Dual Component Oral Composition: Example 10

| First | Concentration | | Concentration |
|---|---|---|---|
| Component | (%) | Second Component | (%) |
| Solvent | 20-50 | Solvent | 20-50 |
| Charcoal | 5-20 | Botanical Activator | 5-30 |
| Whitener | 1-5 | Other ingredients, as | 20-75 |
| | | required | |
| Other | 25-68 | | |
| ingredient, | | | |
| as required | | | |

Dual Component Oral Composition: Example 11

| First | Concentration | | Concentration |
|---|---|---|---|
| Component | (%) | Second Component | (%) |
| Solvent | 20-50 | Solvent | 20-50 |
| Charcoal | 5-20 | Astrocaryum Murumuru | 5-30 |
| Calcium | 1-5 | Other ingredients, as | 20-75 |
| Peroxide | | required | |
| Other | 25-68 | | |
| ingredient, | | | |
| as required | | | |

Dual Component Oral Composition: Example 12

| First Component | Concentration (%) |
|---|---|
| Alkaline Water | 20-50 |
| Calcium Peroxide | 1-5 |
| Activated Charcoal, from Coconuts | 5-20 |
| Hydrated Silica Sident 9 | 5-20 |
| Hydrated Silica Sident 22s | 1-5 |
| Glycerin | 5-20 |
| Xylitol | 5-20 |
| Irish Moss | 0-3 |
| Sodium Coco Sulphate | 0-1 |
| Titanium Dioxide | 0-1 |
| Flavor | 0-1 |

| Second Component | Concentration (%) |
|---|---|
| Alkaline Water | 20-50 |
| Astrocaryum Murumuru | 5-30 |
| Hydrated Silica Sident 9 | 5-20 |
| Hydrated Silica Sident 22s | 1-5 |
| Glycerin | 5-20 |
| Xylitol | 5-20 |
| Irish Moss | 0-3 |
| Sodium Coco Sulphate | 0-1 |
| Titanium Dioxide | 0-1 |
| Flavor | 0-1 |

The oral composition illustrated in Example 12 may be made by the following protocol. This protocol may be adapted to formulate any of the oral compositions contemplated herein. To a clean sanitized vessel with sweep agitation and homogenization, water, sodium coco sulphate, xylitol, glycerin and titanium dioxide was added. This was mixed for 5 minutes between ingredient additions under sweeps (16±2 RPM). Once all ingredients were added at 16±2 RPM, mixing for 30 minutes under vacuum was continued. In a separate vessel, the Irish Moss and Silica was blended. One-third of Sident 9 and ⅓ of Irish Moss was mixed by hand. This was repeated 2 more times in the same container until all Sident 9 and Irish Moss were mixed. The first ⅓ of the pre-mix was added to the main vessel with homogenizer ON under vacuum at 16±2 RPM. This was repeated 2 more times until all of the pre-mix was added. This was then mixed for 10 more minutes.

Sident 22s was added to the main vessel and mixing under vacuum was continued. When the main vessel was uniformly mixed, aloe juice and a flavor were added. Continued mixing (16±2 RPM), under vacuum with sweeps for 30 minutes was undertaken. Once everything was homogenous and had the appearance of a finished toothpaste, Coconut Oil was added at 16±2 RPM with NO Homogenizer under vacuum. Activated charcoal was added to the main vessel and mixed at very slow speed under vacuum. The oral composition had a very light gray color with black specks. The oral composition can then undergo quality control for taste testing and other QC testing. The oral composition may further be packaged into a portable container for filling.

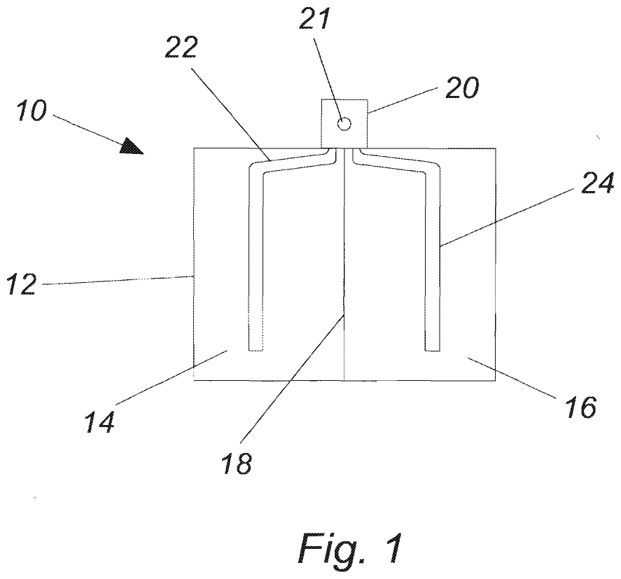

As described above, in use, the plant based activator, in this case the Murumuru, reacts with the whitening agent, illustrated herein as calcium peroxide, and water from saliva, to cause breakdown into calcium and oxygen (O—) free radicals. The free radicals are free aid in whitening of the teeth. When placed in water, calcium peroxide begins to decompose and release oxygen. Murumuru is used to enhance the speed of the process. Preferably, the peroxide is kept separated from the Murumuru, until actual use, i.e. the consumer is ready to brush their teeth. When both components are dispensed onto the toothbrush together, the Murumuru and calcium peroxide combine for the first time, and then start to foam and whiten when the entire mixture comes into contact with water from saliva. To achieve maximum effect, the oral composition in accordance with the invention is preferably stored in a dual composition dispensing unit 10, see FIG. 1. The dual composition dispensing unit 10 comprises a container 12 having a first inner compartment 14 (for storing the first component of the oral composition) and a second inner compartment 16 (for storing the second component of the oral composition). The first inner compartment 14 and the second inner compartment 16 are separated by a barrier 18, such as a wall, preventing the intermixing of any substances stored within the first inner compartment 14 and the second inner compartment 16. A dispensing pump assembly having a nozzle 20 with an opening 21 (shown with one opening, but the nozzle may have two openings) connected to a first inner compartment tube 22 and a second inner compartment tube 24 allows the contents stored within the first inner compartment 14 and the second inner compartment 16 to be dispensed outwardly. The nozzle 20 may be designed to dispense the contents stored within the first inner compartment 14 and the second inner compartment 16 out in a separate manner or as a mixed composition.

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention, and the invention is not to be considered limited to what is shown and described in the specification and any drawings/FIGURES included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A composition to promote oral cavity health comprising:
charcoal;
a whitening agent;
a botanical activator; and
a solvent, an abrasive, a thickening agent, a secondary thickening agent, a humectant, a foaming agent, a non-ionic agent, a flavoring agent, a coloring agent, or combinations thereof.

2. The composition to promote oral cavity health according to claim 1, wherein said botanical actuator is Astrocaryum Murumuru.

3. A composition for delivery to an oral cavity to promote oral cavity health comprising a first component and a second component;
said first component comprising:
charcoal,
a chemical whitener;
said second component comprising:
a botanical activator,
wherein said first component is separated from said second component until delivery of said composition to one or more components of an oral cavity.

4. The composition to promote oral cavity health according to claim 3, wherein said botanical actuator comprises a component of Astrocaryum Murumuru.

5. The composition to promote oral cavity health according to claim 3, wherein said botanical actuator is Murumuru butter.

6. The composition to promote oral cavity health according to claim 3, wherein said first component includes a solvent, an abrasive, a thickening agent, a secondary thickening agent, a humectant, a foaming agent, a non-ionic agent, a flavoring agent, a coloring agent, or combinations thereof.

7. The composition to promote oral cavity health according to claim 3, wherein said second component includes a solvent, an abrasive, a thickening agent, a secondary thickening agent, a humectant, a foaming agent, a non-ionic agent, a flavoring agent, a coloring agent, or combinations thereof.

8. The composition to promote oral cavity health according to claim 3, wherein said charcoal concentration is between about 5% and 20%.

9. The composition to promote oral cavity health according to claim 4, wherein said Astrocaryum Murumuru concentration is between about 5% and 30%.

10. The composition to promote oral cavity health according to claim 5, wherein said Murumuru butter concentration is between 5% and 30%.

11. The composition to promote oral cavity health according to claim 3, wherein said whitener is a peroxide.

12. The composition to promote oral cavity health according to claim 3, wherein said peroxide is calcium peroxide, hydrogen peroxide, or carbamide peroxide.

13. The composition to promote oral cavity health according to claim 3, wherein said charcoal is activated charcoal.

14. The composition to promote oral cavity health according to claim 3, wherein said composition is a toothpaste, gel, or liquid.

15. A dual component oral composition to promote oral cavity health comprising:
a first component comprising:
a solvent, at a concentration of between about 20%-50%;
charcoal, at a concentration of between about 2%-20%;
a whitener, at a concentration of between about 2%-20%;
an abrasive, a thickening agent, a secondary thickening agent, a humectant, a foaming agent, a non-ionic agent, a flavoring agent, a coloring agent, or combinations thereof, at a concentration of about between 25% and 68%; and
a second component comprising:
a solvent, at a concentration of between about 20%-50%;
a botanical activator, at a concentration of between about 5 and 30%;
an abrasive, a thickening agent, a secondary thickening agent, a humectant, a foaming agent, a non-ionic agent, a flavoring agent, a coloring agent, or combinations thereof, at a concentration of about between 20% and 75%,
wherein said first component is separated from said second component until delivery of said composition to one or more components of an oral cavity.

16. The composition to promote oral cavity health according to claim 15, wherein said botanical activator is Astrocaryum Murumuru or Murumuru butter.

17. The composition to promote oral cavity health according to claim 15, wherein said whitener is a peroxide.

18. The composition to promote oral cavity health according to claim 17, wherein said peroxide is calcium peroxide, hydrogen peroxide, or carbamide peroxide.

19. A method for promoting oral cavity health comprising:
providing a dual component oral composition to promote oral cavity health comprising: a first component comprising a solvent, charcoal, a whitener, and at least one of an abrasive, a thickening agent, a secondary thickening agent, a humectant, a foaming agent, a non-ionic agent, a flavoring agent, a coloring agent, or combinations thereof; and a second component comprising a solvent, a botanical activator, and at least one of an abrasive, a thickening agent, a secondary thickening agent, a humectant, a foaming agent, a non-ionic agent, a flavoring agent, a coloring agent, or combinations thereof;
causing said dual component oral composition to be applied to one or more portions of an oral cavity.

20. The method for promoting oral cavity health according to claim 19 further comprising generating free radicals.
